(19) United States
(12) Patent Application Publication
(10) Pub. No.: US 2017/0119703 A1
(43) Pub. Date: May 4, 2017

(54) METHODS OF TREATMENT OF ACNE VULGARIS USING TOPICAL DAPSONE COMPOSITIONS

(71) Applicant: Allergan, Inc., Irvine, CA (US)

(72) Inventors: Kevin S. Warner, Anaheim, CA (US); Ajay P. Parashar, Fairfax, VA (US); Vijaya Swaminathan, Bangalore (IN); Varsha Bhatt, San Francisco, CA (US); Alexandre Kaoukhov, Newport Beach, CA (US)

(21) Appl. No.: 15/343,978

(22) Filed: Nov. 4, 2016

Related U.S. Application Data
(60) Provisional application No. 62/250,763, filed on Nov. 4, 2015.
(60) Provisional application No. 62/299,978, filed on Feb. 25, 2016.

(51) Int. Cl. A61K 31/145; A61K 47/32; A61K 47/14; A61K 47/10

(52) U.S. Cl. CPC A61K 31/145; A61K 47/10; A61K 47/32; A61K 47/14

(57) ABSTRACT

Dapsone compositions can be useful for treating acne. The methods and formulations disclosed herein show efficacy for treating acne vulgaris and/or post inflammatory hyperpigmentation.

### CROSS-REFERENCE TO RELATED APPLICATIONS

This application claims the benefit of U.S. Provisional Application No. 62/250,763, filed on Nov. 4, 2015, and U.S. Provisional Application No. 62/299,978, filed on Feb. 25, 2016, the entire content of both applications are incorporated herein by reference.

### FIELD

The present embodiments relate generally to methods of treatment of acne vulgaris and/or post-inflammatory hyperpigmentation with topical dapsone compositions.

### BACKGROUND

Acne is a group of common skin conditions characterized by the so-called “acneiform” or acne-like skin eruptions, which can be contaminated with bacteria, such as Propionibacterium acnes, and can also be marked by inflammation. Acne tends to occur in the areas of skin where the sebaceous glands are most active, such as the face. Acne is associated with psychological trauma, and, if left untreated, can lead to scar formation and disfigurement.

Classification and the diagnosis of various acne conditions can be complex, and even contradictory. Given this complexity and unpredictability, medication and other therapies, are often developed on a trial-and-error basis in order to determine the most effective course of treatment for a particular patient. The outcome of any particular acne treatment regimen greatly varies from patient to patient, as well as throughout treatment of a particular patient. In addition to the complexity and variability of acne conditions, treatment efficacy can be greatly affected by a patient's compliance with the treatment regimen. Patient compliance during acne treatment may be influenced by side effects, which, for topical medications, commonly include redness, itching, and skin peeling. The complexity of the drug regimen can also negatively affect patient compliance, particularly where two or more different topical medications are prescribed simultaneously. Another factor that negatively affects patient compliance is the cost of a drug regiment, which is considerably higher when multiple medications are prescribed. In some countries, acne is considered a cosmetic problem, and acne treatments are not covered by insurance plans, thus further increasing patient's treatment costs. Certain compositions for treatment of acne are available. Many of the available compositions include one active agent known to have anti-acne activity. Stability of compositions with multiple anti-acne agents can be problematic. Also, these compositions can be difficult to manufacture.

Accordingly, there is a continuing need for compositions and methods used in a treatment of acne, in which topical application is potentially effective. The compositions and methods provided herein address these and other needs in the art.

### SUMMARY

Dapsone, (4,4′-diaminodiphenyl sulfone) is a medicament possessing several beneficial medicinal activities. Dapsone is typically administered as one of the medicinal agents used in the treatment of leprosy. Dapsone and its derivatives are also effective for treatment of bacterial infections, protozoal infections such as malaria, pneumocystis carinii, and plasmonic infections such as toxoplasmosis.

Dapsone is also useful as an anti-inflammatory agent. It has been used to treat skin diseases characterized by the abnormal infiltration of neutrophils, such as Dermatitis herpetiformis, linear IgA dermatosis, pustular psoriasis, pyoderma gangrenosum, acne vulgaris, and Sweet's Syndrome. Examples of dapsone formulations useful in the present application are found in U.S. Pat. No. 9,161,926, which is herein incorporated by reference in its entirety.

Use of topical compositions of dapsone can be problematic. Topical compositions may act as drying agents for the skin. They remove essential oils and natural skin softeners from the skin thus causing it to be dry, itch and crack. Inclusion of exogeneous skin emollients, oils and the like, however, causes phase separation and precipitation of dapsone. Use of typical emulsifiers does not solve the dapsone precipitation owing to the lowered dapsone solubility and conflicting physical characteristics of the phases of the resulting composition. In particular, topical compositions including dapsone and methods are needed that would, for example, exhibit improved effectiveness, reduced side effects, or both, when used in a particular patient with a skin condition. Such improved topical compositions including dapsone and methods of their uses are also needed to improve treatment of patients with acne or suspected acne. The present methods using dapsone formulations can be useful for treating a variety of dermatological conditions. Some useful compositions include dapsone in a polymeric viscosity builder. Some compositions can be adjusted to optimize the dermal delivery profile of dapsone to effectively treat dermatological conditions and improve the efficiency of pharmaceutical products applied to the skin. Diethylene glycol monoethyl ether is a solubilizer for dapsone, thereby allowing compositions to be prepared with increased solubilized concentrations of dapsone. As a result, the compositions described herein are effective in treating dermatological conditions in a subject in need thereof.

In one embodiment, there are provided compositions including dapsone, a first solubilizing agent which is diethylene glycol monoethyl ether, optionally at least one second solubilizing agent, a polymeric viscosity builder, and water, wherein the dapsone is present at a concentration of about 5% w/w to about 10% w/w.

In one embodiment, there are provided compositions including dapsone, a first solubilizing agent which is diethylene glycol monoethyl ether, optionally at least one second solubilizing agent, a polymeric viscosity builder, and water, wherein the dapsone is present at a concentration of about 3% w/w to 8% w/w.

In another embodiment, there are provided methods for treating a dermatological condition. Such methods can be performed, for example, by administering to a subject in need thereof a therapeutically effective amount of a pharmaceutical composition described herein.

In some embodiments, there are provided methods for treating acne vulgaris by administering a 7.5% w/w dapsone formulation at a frequency of once a day. In some embodiments, the methods significantly reduce lesion count in a period of time in the range of two weeks to twelve weeks. In some embodiments, the incidences of adverse events, such as erythema, scaling, dryness, and/or stinging/burning decrease over treatment. In some embodiments the methods result in very few instances (e.g. <1%) of redness, dryness, and peeling of treated skin.

In some embodiments, a method of treating acne vulgaris in a subject in need thereof, includes administering a topical pharmaceutical composition comprising about 7.5% w/w dapsone to the entire face of the subject at a frequency of once a day for a treatment duration effective to improve the acne vulgaris. The treatment duration can be in the range of about 4 weeks to about 12 weeks. The method can be therapeutically effective to reduce the number of lesions on the face of the subject. In some embodiments, the topical pharmaceutical composition does not comprise adapalene. According to an embodiment, the treatment duration is 12 weeks. In some embodiments, the lesions are inflammatory lesions. In some embodiments, the lesions are non-inflammatory lesions. According to some embodiments, the method is effective to reduce the amount of local cutaneous irritation in the subject over the treatment duration.

In some embodiments, the local cutaneous irritation comprises erythema. In some embodiments, the local cutaneous irritation comprises scaling. According to some embodiments, the local cutaneous irritation comprises dryness. In some embodiments, the local cutaneous irritation comprises stinging/burning. In some embodiments, the topical pharmaceutical composition further comprises about 30% w/w diethylene glycol monoethyl ether. In some embodiments, the topical pharmaceutical composition further comprises 4% w/w of a polymeric viscosity builder consisting of acrylamide/sodium acryloyldimethyl taurate copolymer. In some embodiments, the topical pharmaceutical composition further comprises methyl paraben.

### BRIEF DESCRIPTION OF THE FIGURES

FIG. 1 illustrates the mean percent reduction of acne lesions from baseline over time when comparing formulations of the invention to vehicle, when administered at a frequency of once a day.

FIG. 2 is a chart illustrating the local cutaneous irritation profile over time (at baseline, at maximum severity, and at the end of a 12 week treatment regimen) when formulations of the invention were administered at a frequency of once a day to treat acne vulgaris.

### DETAILED DESCRIPTION

It is to be understood that both the foregoing general description and the following detailed description are exemplary and explanatory only and do not restrict the claims. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Some embodiments include compositions and products for treatment of skin conditions and methods of treating skin conditions. The term “skin condition” as used herein encompasses human and animal conditions, disorders, or diseases affecting skin. Such skin conditions include, but are not limited to, conditions involving skin inflammation, conditions involving sebaceous glands and hair follicles, conditions characterized by acneiform symptoms, and conditions involving skin dryness, skin thickening, skin scaling or skin flaking. Skin conditions that can be treated using some compositions, products and methods described herein include, but are not limited to, acne, rosacea, folliculitis, perioral dermatitis, photodamage, skin aging, psoriasis, ichtiosis, atopic dermatitis, treatment of chronic wounds, bed sores, keratosis piralis, scars, including surgical and acne scars, sebaceous cysts, inflammatory dermatoses, post inflammatory hyperpigmentation, eczema, xerosis, pruritis, lichen planus, nodular prurigo, eczema, and miliaria.

The term “acne” as used herein, encompasses skin conditions involving acneiform or acne-like symptoms. For example, a skin condition characterized by follicular eruptions, such as papules and pustules resembling acne, can be categorized as acne. It is to be understood that the term “acne” is not to be limited to diseases and conditions characterized by papules and pustules, but can be characterized by a variety of symptoms. It is also to be understood that a particular patient having acne can be in remission, or the patient's acne can be controlled by continuing treatments, and therefore the patient can exhibit reduced symptoms or be asymptomatic. Nevertheless, continuing treatment of acne can be recommended in such a patient in order to reduce the probability of the return of the acne symptoms.

Symptoms of acne or acne-like conditions include, but are not limited to, the appearance of various skin lesions. The term “lesion” is generally used to denote an infected or diseased patch of skin. A lesion can involve an infected sebaceous gland. Some lesions are more severe than others. Examples of skin lesions are comedones, macules, papules, pustules, nodules and cysts. The term “comedo” (plural “comedones”) is used to describe a sebaceous follicle plugged with dirt, other cells, tiny hairs, or bacteria. Comedones include the so-called “blackheads,” which can also refer to as “open comedones,” which have a spot or a surface that appears black. Comedones also include slightly inflamed, skin colored bumps, as well as “whiteheads,” which have a spot or a surface that appears white. The term “macule” generally refers to a flat spot or area of the skin with a changed color, such as a red spot. The term “pustule” is generally used to refer to an inflamed, pus-filled lesion, or a small inflamed elevation of the skin that is filled with pus. The term “papule” is generally used to refer to a small, solid, usually inflammatory elevation of the skin that does not contain pus. The term “nodule” is generally used to refer to an elevation of a skin that is similar to a papule but is white and dome-shaped. Colloquially, a papule, a pustule or a nodule can be referred to as “a pimple” or “a zit.” The term “cyst” generally refers to an abnormal membranous sac containing a liquid or semi-liquid substance containing white blood cells, dead cells, and bacteria. Cysts can be painful and extend to deeper layers of skin.

In dermatological science and dermatological and cosmetology practice, acne can be classified or categorized into one or more types or categories, according to one or more lines of categorization, such as a predominantly observed type of symptoms, severity of condition or predominant localization. It is to be understood that classification of acne into one of the subtypes does not mean that the characteristics of the classified condition are limited to the symptoms associated with the specific type.

Acne vulgaris is a common form of acne characterized by the appearance of several types of lesions, which may appear together or separately. Individual acne lesions usually last less than two weeks but the deeper papules and nodules may persist for months. Acne vulgaris commonly affects adolescents, but it may also appear, persist or become more severe in adulthood. Acne vulgaris may occur on the face, chest, back and sometimes even more extensively.

Depending on severity, acne can be mild, moderate or severe. Mild acne is generally categorized by the appearance of with blackheads and whiteheads, but can also include papules and pustules. Moderate acne is generally characterized by appearance of more painful, deep-rooted, inflamed lesions, which can result in scarring. Severe acne is characterized by the appearance of deep-rooted inflammatory lesions, including cysts and nodules which can be painful and can produce scarring. Acne conglobata is a category of acne characterized by highly inflammatory cysts that communicate under the skin with abscesses and burrowing sinus tracts.

Some other skin conditions exhibiting acne-like symptoms which can be treated by the compositions and methods described herein are discussed below. Pyoderma faciale, also known as rosacea fulminans, is a condition that appears in females and is characterized by abrupt appearance of inflamed cysts and nodules localized on the face. Rosacea, which can be referred to as acne rosacea, is a condition that can affects both the skin and the eyes and is characterized by redness, bumps, pimples, and, in advanced stages, thickened skin on the nose. In some classification systems, rosacea and acne are considered as separate conditions. Rosacea usually occurs on the face, although the neck and upper chest are also sometimes involved. A mild degree of eye (ocular) involvement occurs in more than fifty percent of people with rosacea. Perioral dermatitis is characterized by the appearance of small tiny papules, pustules, red bumps and scaling with intense itching. It is usually localized to the surrounding area of the mouth and on the chin, or extends to involve the eyelids and the forehead. Gram-negative folliculitis is a bacterial infection characterized by the appearance of pustules and cysts, possibly occurring as a complication resulting from a long term antibiotic treatment of acne vulgaris.

As used herein, the terms “treatment” or “treating” in reference to a skin condition generally mean “having positive effect on a skin condition” and encompass alleviation of at least one symptom of a skin condition, a reduction in the severity of the skin conditions, or delay, prevention, or inhibition of the progression of the skin condition. Treatment need not mean that the condition is totally cured. A composition or a product useful for treatment of a skin condition, or a method of treating a skin condition, needs only to reduce the severity of a skin condition, reduce the severity of symptoms associated therewith, provide improvement to a patient's quality of life, or delay, prevent, or inhibit the onset of symptoms of a skin condition.

### Formulations

In one embodiment, there are provided compositions including dapsone, a first solubilizing agent which is diethylene glycol monoethyl ether, optionally at least one second solubilizing agent, a polymeric viscosity builder, and water, wherein the dapsone is present at a concentration of about 5% w/w to about 10% w/w, about 1% w/w to about 10% w/w, about 3% w/w to about 10% w/w, about 3% w/w to about 8% w/w, about 4% w/w to about 6% w/w, or about 5%. In certain embodiments, dapsone is present in the composition at 5.0%, 5.5%, 6.0%, 6.5%, 7.0%, 7.5%, 8.0%, 8.5%, 9.0%, 9.5%, or 10.0% w/w.

In some embodiments, the polymeric viscosity builder is an acrylamide/sodium acryloyldimethyltaurate copolymer, and further includes isohexadecane, water, and Polysorbate 80. In some embodiments, the polymeric viscosity builder is present at a concentration of about 2% w/w to about 6% w/w. In some embodiments, the polymeric viscosity builder is present at a concentration of about 3% w/w to about 5% w/w. In some embodiments, the polymeric viscosity builder is present in the composition at about 4% w/w. An example of a commercially available polymeric viscosity builder including acrylamide/sodium acryloyldimethyltaurate copolymer is Sepineo P 600, the MSDS of which is incorporated by reference in its entirety.

In some embodiments, diethylene glycol monoethyl ether is present at a concentration of about 25% w/w to about 40% w/w. In some embodiments, diethylene glycol monoethyl ether is present at a concentration of about 30% w/w to about 40% w/w. In some embodiments, diethylene glycol monoethyl ether is present at a concentration of about 35% w/w to about 40% w/w.

In some embodiments, diethylene glycol monoethyl ether is present at a concentration of about 10% w/w to about 40% w/w, about 20% w/w to about 30% w/w, or about 25% w/w. In some embodiments, diethylene glycol monoethyl ether is present at a concentration of about 30% w/w.

In some embodiments, the second solubilizing agent is selected from alcohols, glycols, esters, ethers, or silicones. Such second solubilizing agents include, but are not limited to, PEG 400, lactic acid, dimethyl isosorbide, propylene glycol, propylene carbonate, hexylene glycol, isostearyl alcohol, benzyl alcohol, diethyl sebacate, and ethanol.

In certain embodiments, the second solubilizing agent is propylene glycol. In some embodiments, propylene glycol is present at a concentration of about 2% w/w to 8% w/w. In some embodiments, propylene glycol is present at a concentration of about 3% w/w to 7% w/w. In some embodiments, propylene glycol is present in the composition at about 5% w/w.

In certain embodiments, the second solubilizing agent is propylene carbonate. In some embodiments, propylene carbonate is present at a concentration of about 2% w/w to 8% w/w. In some embodiments, propylene carbonate is present at a concentration of about 3% w/w to 7% w/w. In some embodiments, propylene carbonate is present in the composition at about 5% w/w.

In certain embodiments, the second solubilizing agent is ethanol. In some embodiments, ethanol is present at a concentration of about 1% w/w to about 5% w/w. In some embodiments, ethanol is present at a concentration of about 2% w/w to about 4% w/w. In some embodiments, ethanol is present in the composition at about 3% w/w.

In some embodiments, the compositions further include methyl paraben.

In other embodiments, the compositions further include carbomer homopolymer type C. In some embodiments, carbomer homopolymer type C is present at a concentration of about 0.7% w/w to about 1.5% w/w. In other embodiments, carbomer homopolymer type C is present at a concentration of about 0.85% w/w to about 1.0% w/w.

In some embodiments, the compositions further include a neutralizing agent. In certain embodiments, the neutralizing agent is an ionic or amine buffer. In certain embodiments, the neutralizing agent is sodium hydroxide or triethanolamine. Use of a neutralizing agent results in compositions typically having a pH from 5.5 to 6.5.

In some embodiments, the compositions further include a chelating agent. In some embodiments, the chelating agent is ethylene diamine tetraacetic acid (EDTA). EDTA is typically present in the compositions from about 0.02% w/w to about 0.04% w/w. In certain embodiments, EDTA is present in the compositions at about 0.03% w/w.

Compositions described herein are typically in the form of a gel, an emulsion, a cream, a liquid, a paste, a lotion, a nanoemulsion, a microemulsion, a reverse emulsion, or a liposomal cream. For example, in an embodiment, the composition can be a 7.5% w/w dapsone gel contained in a pump dispenser.

### Methods of Treatment

According to some embodiments, methods of treating acne vulgaris are provided using the dapsone formulations described herein.

In an embodiment, a patient having acne vulgaris can perform a treatment regimen for a period of time effective to improve the acne vulgaris. The regimen can include applying a dapsone gel formulation as described herein at a frequency of once a day to the face of the patient. In some embodiments the regimen can include applying a 7.5% w/w dapsone gel formulation as described herein at a frequency of once a day to the face of the patient. In some embodiments the regimen can include applying a gel formulation comprising about 7.5% w/w dapsone, about 40% w/w diethylene glycol monoethyl ether, and about 4% acrylamide/sodium acryloyldimethyltaurate copolymer based thickener as described herein at a frequency of once a day to the face of the patient.

According to some embodiments, the treatment regimen can be performed at a sufficient frequency for a period of time effective to improve the acne vulgaris. In some embodiments, the treatment regimen can be performed only once daily. When the treatment regimen is performed once daily, it can be performed at various times such as at night or in the morning.

In some embodiments, the treatment regimen can be performed for a treatment duration effective to improve the acne vulgaris. In some embodiments, the treatment duration effective to improve the acne vulgaris can be about 12 weeks. The treatment duration effective to improve the acne vulgaris can be about 4 weeks, about 8 weeks, about 10 weeks, and the like. According to some embodiments, the treatment duration effective to improve the acne vulgaris can be about 12 weeks or more, about 10 weeks or more, about 8 weeks or more, about 4 weeks or more, and the like. In some embodiments, the treatment duration effective to improve the acne vulgaris can be in the range of about 2 weeks to about 12 weeks. In some embodiments, the treatment duration effective to improve the acne vulgaris can be in the range of about 4 weeks to about 12 weeks. In some embodiments, the treatment duration effective to improve the acne vulgaris can be in the range of about 8 weeks to about 12 weeks. According to some embodiments, the treatment duration effective to improve the acne vulgaris can be determined by a patient's physician.

In some embodiments, an improvement in acne vulgaris can include a reduction in the severity of a patient's acne vulgaris. For example, an improvement in acne vulgaris can, for example, include a reduction in the number of inflammatory and/or non-inflammatory lesions, comedones, papules/pustules or nodulocystic lesions present on the face of the patient with acne vulgaris. In some embodiments, improvement can be present where a patient's nodules change from inflammatory to non-inflammatory. According to some embodiments, an improvement in acne vulgaris can include a reduction of the severity of the acne vulgaris to clear (e.g., no or nearly no evidence of acne vulgaris) or almost clear (e.g. rare non-inflammatory lesions present, with rare non-inflamed papules) as assessed by a physician and/or self-assessed by the patient.

A topical gel formulation can be provided as described above. For example, in an embodiment, a topical gel formulation can be provided comprising dapsone, the dapsone being present in the topical gel formulation in an amount of about 7.5% by weight, based on the total weight of the topical gel formulation.

In some embodiments, the topical dapsone gel formulation can be applied to the face of a patient having acne vulgaris. According to some embodiments, the topical dapsone gel formulation can be applied to the entire face of the patient. After the topical dapsone gel formulation is applied to the patient's face, the topical dapsone gel formulation can be rubbed into the entire face of the patient. In some embodiments, the topical dapsone gel formulation can be rubbed into the entire face of the patient except for the eyes, mouth, and areas immediately surrounding the eyes and/or mouth. In some embodiments, the topical dapsone gel formulation may only be applied to those areas of the face exhibiting the symptoms of acne.

According to some embodiments, the topical dapsone gel formulation can be left on the face for an extended period of time after it is applied. In such embodiments, a patient should not bathe or shower during that extended period of time. In some embodiments, the extended period of time can be about 8 hours or more, about 6 hours or more, about 4 hours or more, and the like.

### Post Inflammatory Hyperpigmentation

According to some embodiments, methods of treating post-inflammatory hyperpigmentation are provided using the dapsone formulations described herein. Post-inflammatory hyperpigmentation (PIH) is a condition in which an injury or inflammation to the skin causes increased pigment production. PIH occurs in darker-skinned individuals (e.g. Fitzpatrick type 5 or 6) and can be difficult to treat. The most common cause of PIH is acne, but it also can result from psoriasis, a burn, or an injury. In some embodiments, the PIH treated is caused by acne vulgaris.

In an embodiment, a patient having PIH can perform a treatment regimen for a period of time effective to improve the PIH. The regimen can include applying a dapsone gel formulation as described herein at a frequency of once a day to the face of the patient. In some embodiments the regimen can include applying a 7.5% w/w dapsone gel formulation as described herein at a frequency of once a day to the face of the patient. In some embodiments the regimen can include applying a gel formulation comprising about 7.5% w/w dapsone, about 40% w/w diethylene glycol monoethyl ether, and about 4% of an acrylamide/sodium acryloyldimethyltaurate copolymer based thickener as described herein at a frequency of once a day to the face of the patient.

According to some embodiments, the treatment regimen can be performed at a sufficient frequency for a period of time effective to improve a patient's post inflammatory hyperpigmentation. In some embodiments, the treatment regimen can be performed only once daily. When the treatment regimen is performed once daily, it can be performed at various times such as at night or in the morning.

In some embodiments, the treatment regimen can be performed for a treatment duration effective to improve the PIH. In some embodiments, the treatment duration effective to improve the PIH can be about 12 weeks. The treatment duration effective to improve the PIH can be about 4 weeks, about 8 weeks, about 10 weeks, and the like. According to some embodiments, the treatment duration effective to improve the PIH can be about 12 weeks or more, about 10 weeks or more, about 8 weeks or more, about 4 weeks or more, and the like. In some embodiments, the treatment duration effective to improve the PIH can be in the range of about 2 weeks to about 12 weeks. In some embodiments, the treatment duration effective to improve the PIH can be in the range of about 4 weeks to about 12 weeks. In some embodiments, the treatment duration effective to improve the PIH can be in the range of about 8 weeks to about 12 weeks. According to some embodiments, the treatment duration effective to improve the PIH can be determined by a patient's physician.

In some embodiments, an improvement in PIH can include a reduction in the severity of a patient's PIH. For example, an improvement in PIH can, for example, include a reduction in the number of dark spots and/or a lightening of the dark spots present on the face of the patient with PIH. In some embodiments, an improvement in PIH can include the total clearing of dark spots on the face of the patient.

In some embodiments, the methods of treatment can be effective to treat a patient having both acne vulgaris and PIH. For example, a patient with acne vulgaris could use the formulations and methods described above to treat their acne, then treat the PIH resulting from the acne vulgaris with the same formulations and methods.

### EMBODIMENTS

The following example dapsone formulation embodiments are specifically contemplated herein.

### Embodiment 1

A composition comprising dapsone, a first solubilizing agent which is diethylene glycol monoethyl ether, optionally at least one second solubilizing agent, a polymeric viscosity builder, and water, wherein the dapsone is present in the composition at a concentration of about 3% w/w to about 10% w/w.

### Embodiment 2

The composition of embodiment 1, wherein the diethylene glycol monoethyl ether is present at a concentration of about 10% w/w to about 40% w/w.

### Embodiment 3

The composition of embodiment 1, wherein the diethylene glycol monoethyl ether is present at a concentration of about 20% w/w to about 30% w/w.

### Embodiment 4

The composition of embodiment 1, wherein the diethylene glycol monoethyl ether is present in the composition at a concentration of about 25% w/w.

### Embodiment 5

The composition of embodiment 1 wherein the second solubilizing agent is selected an alcohol, a glycol, an ester, or an ether.

### Embodiment 6

The composition of embodiment 1, wherein the second solubilizing agent is PEG 400, lactic acid, dimethyl isosorbide, propylene glycol, propylene carbonate, hexylene glycol, isostearyl alcohol, diethyl sebacate, or ethanol.

### Embodiment 7

The composition of embodiment 6, wherein the second solubilizing agent is propylene glycol.

### Embodiment 8

The composition of embodiment 7, wherein the propylene glycol is present in the composition at a concentration of about 5% w/w.

### Embodiment 9

The composition of embodiment 6, wherein the second solubilizing agent is propylene carbonate.

### Embodiment 10

The composition of embodiment 9, wherein the propylene carbonate is present in the composition at a concentration of about 5% w/w.

### Embodiment 11

The composition of embodiment 6, wherein the second solubilizing agent is ethanol.

### Embodiment 12

The composition of embodiment 11, wherein the ethanol is present in the composition at a concentration of about 3% w/w.

### Embodiment 13

The composition of embodiment 1, wherein the polymeric viscosity builder comprises an acrylamide/sodium acryloyldimethyltaurate copolymer.

### Embodiment 14

The composition of embodiment 1, wherein the polymeric viscosity builder is present at a concentration of about 2% w/w to about 6% w/w.

### Embodiment 15

The composition of embodiment 1, wherein the polymeric viscosity builder is present at a concentration of about 4% w/w.

### Embodiment 16

The composition of embodiment 1, further comprising methyl paraben.

### Embodiment 17

The composition of embodiment 1, further comprising Carbomer interpolymer type A, Carbomer interpolymer type B, or Carbomer Homopolymer Type C.

### Embodiment 18

The composition of embodiment 17, wherein the Carbomer Homopolymer Type C is present at a concentration of about 0.7% w/w to about 1.5% w/w.

### Embodiment 19

The composition of embodiment 17, wherein the Carbomer Homopolymer Type C is present at a concentration of about 0.85% w/w to about 1.5% w/w.

### Embodiment 20

The composition of embodiment 17, wherein the Carbomer interpolymer Type A is present at a concentration of about 1% w/w to 2% w/w.

### Embodiment 21

The composition of embodiment 17, wherein the Carbomer interpolymer Type B is present at a concentration of about 0.1% w/w to about 0.5% w/w.

### Embodiment 22

The composition of embodiment 1, further comprising a neutralizing agent.

### Embodiment 23

The composition of embodiment 22 wherein the neutralizing agent is NaOH or triethanolamine.

### Embodiment 24

The composition of embodiment 1 further comprising a chelating agent.

### Embodiment 25

The composition of embodiment 24, wherein the chelating agent is ethylene diamine tetraacetic acid.

### Embodiment 26

The composition of embodiment 25, wherein the ethylene diamine tetraacetic acid is present at a concentration of about 0.02% w/w to about 0.04% w/w.

### Embodiment 27

The composition of embodiment 25, wherein the ethylene diamine tetraacetic acid is present in the composition at about 0.03% w/w.

### Embodiment 28

The composition of embodiment 1 wherein the composition is in the form of a gel, a suspension, an emulsion, a cream, a liquid, a paste, a lotion, a nanoemulsion, a microemulsion, a reverse emulsion, or a liposomal cream.

### Embodiment 29

A method for treating a dermatological condition comprising administering to a subject in need thereof a therapeutically effective amount of a composition of embodiment 1.

### Embodiment 30

The method of embodiment 29 wherein the condition is acne vulgaris, rosacea, atopic dermatitis, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, eczema, or miliaria.

### Embodiment 31

The method of embodiment 30 wherein the condition is acne vulgaris.

### Embodiment 32

The composition of embodiment 1, 2, 3, 4, 30, or 31, wherein the second solubilizing agent is selected an alcohol, a glycol, an ester, or an ether.

### Embodiment 33

The composition of embodiment 1, 2, 3, 4, 30, 31, or 32, wherein the second solubilizing agent is PEG 400, lactic acid, dimethyl isosorbide, propylene glycol, propylene carbonate, hexylene glycol, isostearyl alcohol, diethyl sebacate, or ethanol.

### Embodiment 34

The composition of embodiment 33, wherein the second solubilizing agent is propylene glycol.

### Embodiment 35

The composition of embodiment 34, wherein the propylene glycol is present in the composition at a concentration of about 5% w/w.

### Embodiment 36

The composition of embodiment 33, wherein the second solubilizing agent is propylene carbonate.

### Embodiment 37

The composition of embodiment 36, wherein the propylene carbonate is present in the composition at a concentration of about 5% w/w.

### Embodiment 38

The composition of embodiment 33, wherein the second solubilizing agent is ethanol.

### Embodiment 39

The composition of embodiment 38, wherein the ethanol is present in the composition at a concentration of about 3% w/w.

### Embodiment 40

The composition of embodiment 1, 2, 3, 4, 30, 31, 32, 33, 34, 35, 36, 37, 38, or 39, wherein the polymeric viscosity builder comprises an acrylamide/sodium acryloyldimethyltaurate copolymer.

### Embodiment 41

The composition of embodiment 1, 2, 3, 4, 30, 31, 32, 33, 34, 35, 36, 37, 38, 39, or 40 wherein the polymeric viscosity builder is present at a concentration of about 2% w/w to about 6% w/w.

### Embodiment 42

The composition of embodiment 41, wherein the polymeric viscosity builder is present at a concentration of about 4% w/w.

### Embodiment 43

The composition of embodiment 1, 2, 3, 4, 30, 31, 32, 33, 34, 35, 36, 37, 38, 39, 40, 41, or 42, further comprising methyl paraben.

### Embodiment 44

The composition of embodiment 1, 2, 3, 4, 30, 31, 32, 33, 34, 35, 36, 37, 38, 39, 40, 41, 42, or 43 further comprising Carbomer interpolymer type A, Carbomer interpolymer type B, or Carbomer Homopolymer Type C.

### Embodiment 45

The composition of embodiment 44, wherein the Carbomer Homopolymer Type C is present at a concentration of about 0.7% w/w to about 1.5% w/w.

### Embodiment 46

The composition of embodiment 44, wherein the Carbomer Homopolymer Type C is present at a concentration of about 0.85% w/w to about 1.5% w/w.

### Embodiment 47

The composition of embodiment 44, wherein the Carbomer interpolymer Type A is present at a concentration of about 1% w/w to 2% w/w.

### Embodiment 48

The composition of embodiment 44, wherein the Carbomer interpolymer Type B is present at a concentration of about 0.1% w/w to about 0.5% w/w.

### Embodiment 49

The composition of embodiment 1, 2, 3, 4, 30, 31, 32, 33, 34, 35, 36, 37, 38, 39, 40, 41, 42, 43, 44, 45, 46, 47, or 48 further comprising a neutralizing agent.

### Embodiment 50

The composition of embodiment 49 wherein the neutralizing agent is NaOH or triethanolamine.

### Embodiment 51

The composition of embodiment 1, 2, 3, 4, 30, 31, 32, 33, 34, 35, 36, 37, 38, 39, 40, 41, 42, 43, 44, 45, 46, 47, 48, 49, or 50 further comprising a chelating agent.

### Embodiment 52

The composition of embodiment 51, wherein the chelating agent is ethylene diamine tetraacetic acid.

### Embodiment 53

The composition of embodiment 52, wherein the ethylene diamine tetraacetic acid is present at a concentration of about 0.02% w/w to about 0.04% w/w.

### Embodiment 54

The composition of embodiment 52, wherein the ethylene diamine tetraacetic acid is present in the composition at about 0.03% w/w.

### Embodiment 55

The composition of embodiment 1, 2, 3, 4, 30, 31, 32, 33, 34, 35, 36, 37, 38, 39, 40, 41, 42, 43, 44, 45, 46, 47, 48, 49, 50, 51, 52, 53, or 54 wherein the composition is in the form of a gel, a suspension, an emulsion, a cream, a liquid, a paste, a lotion, a nanoemulsion, a microemulsion, a reverse emulsion, or a liposomal cream.

### Embodiment 56

A method for treating a dermatological condition comprising administering to a subject in need thereof a therapeutically effective amount of a composition of embodiment 1, 2, 3, 4, 30, 31, 32, 33, 34, 35, 36, 37, 38, 39, 40, 41, 42, 43, 44, 45, 46, 47, 48, 49, 50, 51, 52, 53, 54 or 55.

### Embodiment 57

The method of embodiment 56 wherein the condition is acne vulgaris, rosacea, atopic dermatitis, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, eczema, or miliaria.

### Embodiment 58

The method of embodiment 56 wherein the condition is acne vulgaris.

The following examples are intended only to illustrate the some embodiments and should in no way be construed as limiting the claims.

### EXAMPLES

### Example 1

Table 1 lists two formulations (containing equivalent levels of diethylene glycol monoethyl ether) that show the impact of acrylamide/sodium acryloyldimethyltaurate copolymer based thickener on dapsone particle size.

TABLE 1
Formulations Tested For Dapsone Crystal Size

| Formulation # | ENA | ENC |
|---|---|---|
| Dapsone | 7.5 | 7.5 |
| Diethylene glycol monoethyl ether | 30 | 30 |
| Carbomer homopolymer type C. | — | 1 |
| acrylamide/sodium | 4 | — |
| acryloyldimethyltaurate copolymer | | |
| based thickener | | |
| Methyl paraben | 0.2 | 0.2 |
| pH adjusting solution | pH 5.5-7 | pH 5.5-7 |
| Purified Water | Q.S 100 | Q.S 100 |

### Example 2

Anti-oxidants and chelating agents such as sodium metabisulfite, citric acid and EDTA were added to formulations to help slow down or completely stop any impurity formation. Table 2 presents the composition of formulations tested. Formulation A7 with sodium metabisulfite minimized the intensity of yellow color caused by the increased solubility of dapsone in diethylene glycol monoethyl ether and maintained the low color intensity over time at accelerated condition (400 C).

TABLE 2
Compositions Tested containing Antioxidants or Chelating Agents

| Composition # | A5 | A6 | A7 |
|---|---|---|---|
| Dapsone | 7.5 | | |
| Diethylene glycol monoethyl | 35 | 40 | 35 |
| ether | | | |
| carbomer homopolymer type C | 1.25 | — | 1.25 |
| Acrylamide/sodium | — | 4 | — |
| acryloyldimethyltaurate | | | |
| copolymer emulsion | | | |

| | | |
|---|---|---|
| EDTA | 0.05 | — |
| Anhydrous Citric Acid | 0.1 | — |
| Sodium Metabisulfite | — | 0.2 |
| Methyl paraben | 0.17 | 0.2 |
| Propyl paraben | 0.03 | — |

| | |
|---|---|
| NaOH/pH adjusting solution | pH 5.5-6.5 |
| Purified Water | Q.S 100 |

### Example 3

Additional example compositions contemplated for use as described herein are set forth in Table 3 below.

TABLE 3
Additional examples containing alternate neutralizer

| | % w/w | | | | | |
|---|---|---|---|---|---|---|
| Materials | 5-1 | 5-2 | 5-3 | 5-4 | 5-5 | 5-6 |
| Dapsone | 7.5 | | | | | |
| Diethylene glycol monoethyl ether | 30 | 35 | 40 | 30 | 40 | 25 |

| | |
|---|---|
| carbomer homopolymer type C | 1 |
| Methylparaben | 0.2 |
| Triethanolamine (TEA) Q.S. | pH 5.5-6.5 |
| Hydrochloric Acid Q.S | pH 5.5-6.5 |
| Purified Water | q.s.a.d. 100 |

### Example 4

Additional example compositions contemplated for use as described herein are set forth in Table 4 below.

TABLE 4
Additional examples (containing co-solvents, stabilizer and
alternate thickener)

| | % w/w | | | | | |
|---|---|---|---|---|---|---|
| Materials | 6-1 | 6-2 | 6-3 | 6-4 | 6-5 | 6-6 |
| Dapsone | 7.5 | | 10 | 7.5 | | |
| Diethylene glycol monoethyl ether | 25 | 35 | 35 | 25 | 30 | 40 |
| Propylene glycol | 5 | | | | | |
| Propylene Carbonate | 5 | | | | | |
| Ethanol (absolute) | 3 | | | — | 3 | |
| EDTA | 0.03 | | | | | |
| Carbomer Interpolymer Type A | — | | | 1.5 | | |
| Carbomer Interpolymer Type B | — | | | 0.3 | | |
| Acrylamide/sodium | 4 | | | — | | 4 |
| acryloyldimethyltaurate | | | | | | |
| copolymer emulsion | | | | | | |

| Methyl Paraben | 0.2 | |
|---|---|---|
| Triethanolamine | — | Q.S. pH 5.5-6.5 |

| | |
|---|---|
| Purified Water | q.s.a.d. 100 |

### Example 5

### Clinical Studies

Two Phase 3 clinical studies investigated the safety and efficacy of the use of a 7.5% w/w dapsone gel compared to vehicle for the treatment of acne vulgaris.

The formulations used in the studies included the formulation elements listed below:

| | Dapsone | Vehicle |
|---|---|---|
| Formulation | gel % (w/w) | % (w/w) |
| Dapsone | 7.5 | — |
| Diethylene glycol monoethyl ether | 30 | 30 |
| acrylamide/sodium | 4 | 4 |
| acryloyldimethyltaurate copolymer | | |
| based thickener | | |
| Methyl paraben | 0.2 | 0.2 |
| pH adjusting solution | pH 5.5-7 | pH 5.5-7 |
| Purified Water | Q.S 100 | Q.S 100 |

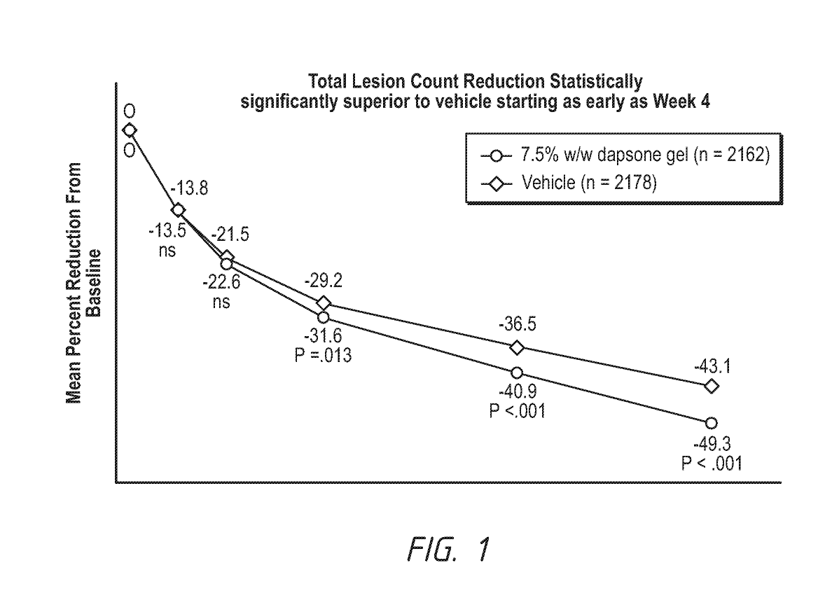

A total of 4340 patients were enrolled in the Phase 3 studies. The patients enrolled in the studies were 12 years and older with 20-50 inflammatory lesions and about 30-100 noninflammatory lesions. Patients were randomized in a 1:1 ratio by study coordinators to one of two treatment groups. In the first group, patients administered the topical dapsone gel formulation containing 7.5% w/w dapsone to their entire face once a day. In the second group, patients administered the vehicle formulation once a day. Patients were treated for twelve weeks. The patients were assessed by a physician throughout the trial for reduction in inflammatory and non-inflammatory lesions and adverse events at weeks 0, 1, 2, 4, 8 and 12. The overall reduction in lesions in the pooled results of the two studies is illustrated in FIG. 1.

Surprisingly, the mean percent reduction in total lesions was statistically significantly superior to vehicle, starting at week 4 and continuing through to week 12. Both inflammatory and non inflammatory were reduced significantly. Specifically, the mean percentage of total lesion reduction in patients administering the topical dapsone gel formulation containing 7.5% w/w dapsone to their entire face once a day was −31.6% at week 4, −40.9% at week 8, and −49.3% at week 12. At week 12, inflammatory lesions were reduced by 15.8 lesions (54.6%; n=2162) vs 13.9 lesions with vehicle (48.1%; n=2178), and non-inflammatory lesions were reduced by 20.7 lesions (45.1%) vs 18.0 lesions with vehicle (39.4%). The Global Acne Assessment Score “GAAS” success rate in patients was 29.8% (n=2162) vs 21.1% with vehicle (n=2178).

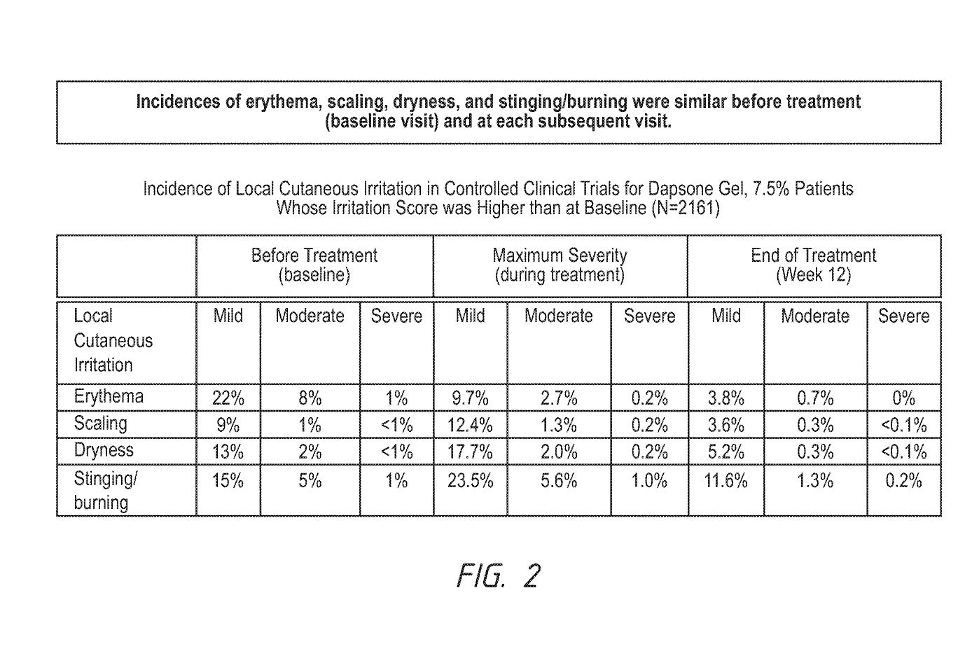

The patients were also assessed for erythema, scaling, dryness, and stinging/burning throughout the trials. A chart showing the incidence of local cutaneous irritation in patients whose irritation score was high than at baseline is shown in FIG. 2. Surprisingly, the amount and severity of erythema, scaling, dryness, and stinging/burning was reduced over the treatment period (between baseline and end of treatment at 12 weeks). Also, during the study, the formulation was extremely well tolerated. Less than 1% of total patients experienced redness, dryness and peeling of the treated skin.

While improvements in acne severity were significant for all subgroups of age, gender and race, improvements were even greater in adults compared to adolescents and in females compared to males.

During the same trials, it was also discovered that a statistically significant resolution of postinflammatory hyperpigmentation in patients. Specifically, patients with darker skin (Fitzpatrick type 5 or 6) experienced accelerated resolution of postinflammatory hyperpigmentation caused by acne lesions. A reduction in the number of dark spots was observed, and a significant number of patients reported no dark spots at week 12 of treatment.

While this some embodiments have been described with respect to these specific examples, it is understood that other modifications and variations are possible without departing from the spirit of the invention. Each and every reference identified herein is incorporated by reference in its entirety.

Attached herewith is the prescribing information for ACZONE® Gel, 7.5%, which is an embodiment of the formulations and methods of treatment described herein.

What is claimed is:

1. A method of treating acne vulgaris in a subject in need thereof, the method comprising:
administering a topical pharmaceutical composition comprising about 7.5% w/w dapsone to the entire face of the subject at a frequency of once a day for a treatment duration effective to improve the acne vulgaris;
wherein the treatment duration is in the range of about 4 weeks to about 12 weeks; and
wherein the method is therapeutically effective to reduce the number of lesions on the face of the subject.

2. The method of claim 1, wherein the topical pharmaceutical composition does not comprise adapalene.

3. The method of claim 2, wherein the treatment duration is 12 weeks.

4. The method of claim 1, wherein the lesions comprise inflammatory lesions.

5. The method of claim 1, wherein the lesions comprise non-inflammatory lesions.

6. The method of claim 1, wherein the method is effective to reduce the amount of local cutaneous irritation in the subject over the treatment duration.

7. The method of claim 6, wherein the local cutaneous irritation comprises erythema.

8. The method of claim 6, wherein the local cutaneous irritation comprises scaling.

9. The method of claim 6, wherein the local cutaneous irritation comprises dryness.

10. The method of claim 6, wherein the local cutaneous irritation comprises stinging/burning.

11. The method of claim 1, wherein the topical pharmaceutical composition further comprises about 30% w/w diethylene glycol monoethyl ether.

12. The method of claim 11, wherein the topical pharmaceutical composition further comprises 4% w/w of a polymeric viscosity builder comprising acrylamide/sodium acryloyldimethyl taurate copolymer.

13. The method of claim 12, wherein the topical pharmaceutical composition further comprises methyl paraben.

14. A method of treating postinflammatory hyperpigmentation in a subject in need thereof, the method comprising:
administering a topical pharmaceutical composition comprising about 7.5% w/w dapsone to the entire face of the subject at a frequency of once a day for a treatment duration effective to reduce the number of dark spots on the face of the subject;
wherein the treatment duration is in the range of about 4 weeks to about 12 weeks; and
wherein the method is therapeutically effective to improve the postinflammatory hyperpigmentation.

15. The method of claim 14, wherein the topical pharmaceutical composition does not comprise adapalene.

16. The method of claim 15, wherein the treatment duration is 12 weeks.

17. The method of claim 16, wherein the dark sports are completely eliminated.

18. The method of claim 14, wherein the topical pharmaceutical composition further comprises about 30% w/w diethylene glycol monoethyl ether.

19. The method claim 14, wherein the topical pharmaceutical composition further comprises 4% w/w of a polymeric viscosity builder comprising acrylamide/sodium acryloyldimethyl taurate copolymer.

20. The method of claim 19, wherein the topical pharmaceutical composition further comprises methyl paraben.
